(19) United States
(12) Patent Application Publication
(10) Pub. No.: US 2010/0232725 A1
(43) Pub. Date: Sep. 16, 2010

(54) TEMPERATURE ARTIFACT CORRECTION

(75) Inventors: Johannes Albert Luijendijk, Veldhoven (NL); Heidrun Steinhauser, Eindhoven (NL); Bernd Menser, Neu-Moresnet (BE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/294,505

(22) PCT Filed: Mar. 15, 2007
(86) PCT No.: PCT/IB2007/050887

§ 371 Date: Sep. 25, 2008

(30) Foreign Application Priority Data
Mar. 29, 2006 (EP) 06111943.4

(51) Int. Cl. G06K 9/40

(52) U.S. Cl. 382/274; 382/275; 382/275

(57) ABSTRACT

A system and method of generating a template of at least one artifact for use in image correction is disclosed. An image containing the artifact is generated using at least two homogeneous exposures, each generated at a different detector operating temperature. The local variance of grey values at each pixel position in the image is calculated. Each pixel in the image is then classified. A binary image is generated based on the classification. The template is then formed based on both the binary image and the image data containing the artifact.

The invention relates to the field of x-ray imaging and more particularly to the field of digital imaging using flat panel detectors.

Imaging techniques have traditionally used photographic film as image data receptors to capture image data from desired regions of interest. In recent times, there has been a significant shift in the technology associated with image receptors, evolving from the use of analog technology to the use of digital technology.

Flat panel detector (FPD) technology finds widespread implementation in the field of imaging and is effecting the transition from analog imaging to digital imaging. Apart from being able to acquire imaging data quickly, FPDs are widely known for their compact size and longer service life when compared to previously available technology such as the image intensifier. Besides finding an increasing use in human medicine, other fields, including dentistry, nondestructive testing, and veterinary medicine, are seizing on the advantages offered by FPDs, such as being able to dispense with time-consuming processing, and cost of storage of film.

FPDs use a scintillator in conjunction with one or more photo detectors. When radiation strikes the scintillator, typically comprising Cesium Iodide (CsI) as the active material, the scintillator converts the incident radiation to photons of light. When these photons strike the photo detector, a number of electrons is generated that is proportional to the number of photons. Thus, image information is converted into electrical signals for further processing.

FPDs produce poor quality images because of sensitivity variations. This may be due to a variety of reasons, such as the presence of air-bubbles between the FPD and the scintillator, temperature variation, defects in manufacture and assembly of the FPDs. In the case of FPDs having active cooling i.e., when the temperature is kept constant, variations in local pixel intensities can be corrected by commonly known gain correction methods. However, in the case of FPDs not having an active cooling mechanism, image artifacts, caused by poor and/or unstable optical contact between the photodiodes of an FPD and the scintillator can drift with the temperature of the detector.

When the temperature of the detector is different from the temperature used during gain calibration, this commonly used gain correction method become ineffective in correcting the image artifacts. The drifting sensitivity results in poor image quality. Therefore, it would be advantageous to have an imaging procedure that corrects for the temperature dependent effects.

Accordingly, a system and method of generating a template of at least one artifact for use in image correction is described herein. An image containing the artifact is generated using at least two homogeneous exposures, each generated at a different detector operating temperature. The local variance of grey values at each pixel position in the image is calculated. Each pixel in the image is then classified. A binary image is generated based on the classification. The template is then formed based on both the binary image and the image data containing the artifact.

Further, a system and method of image correction is described herein. A template of at least one region of contiguous pixels exhibiting the artifact in an image that needs correction is used. A scalar product based on the template and the image is generated. The image is then corrected based on the match determined based on the scalar product.

Further, a tangible, computer readable media containing code to implement the methods described above is disclosed.

Various features, aspects, and advantages will be apparent when the following detailed description is read with reference to the drawings, wherein:

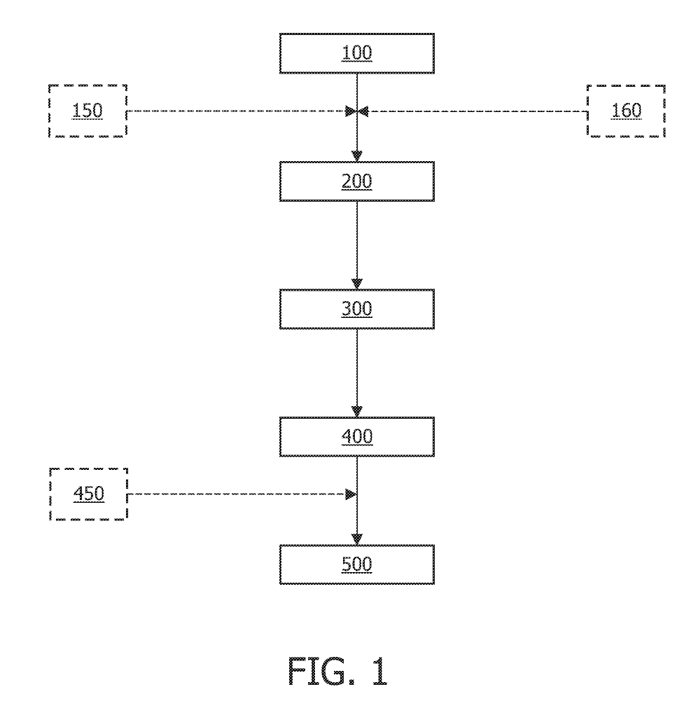

FIG. 1 is a flowchart representing an implementation of a method of generating a template of at least one artifact for use in image correction; and

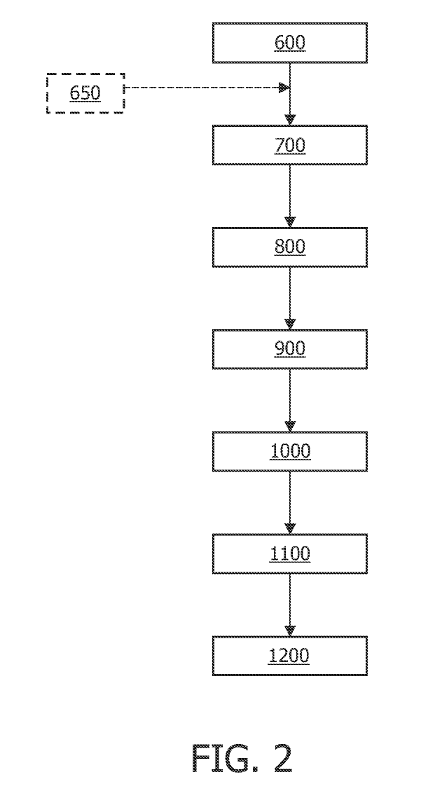

FIG. 2 is a flowchart representing an implementation of a method of image correction.

In the description herein, pixellisation is the term given for image artifacts that are caused by poor and unstable optical contact between the photodiodes of a FPD and the scintillator. The optical contact changes as a function of temperature. Pixellisation lowers detector sensitivity. The spatial variance of sensitivity is high by the nature of the artifact. Temperature also has an effect on the pixellisation because spatial variance of sensitivity drifts as a function of temperature. The sensitivity distribution in a pixellised region i.e., a region exhibiting pixellisation, behaves similar to that of random noise signals. Another feature of pixellisation is that the change in average intensity, as a function of temperature, in a pixellised region is similar and comparable to the average intensity change of a non-pixellised region i.e., a region not exhibiting pixellisation. This means that overall change in sensitivity inside and outside of the pixellisation region is similar. However, the spatial variance of sensitivity drift is higher in pixellisation regions. Sensitivity drift may be defined as ratio of sensitivity at a certain temperature and sensitivity at a different temperature. Pixellisation deteriorates the image in the high end of the spatial frequency spectrum.

Higher spatial variance, i.e. the stochastic (random) appearance of sensitivity distribution in a pixellised region, allows pixellisation to be easily distinguishable from the clinical information in the clinical image. The random appearance of sensitivity distribution means that the correction method will hardly affect the image contents.

In one embodiment, a pixellisation template needs to be generated before image correction can be performed on a sample image. This template serves as a fingerprint for correcting pixellisation in the sample image. Therefore, the act of generating a template can be considered as a calibration stage for the image correction. FIG. 1 shows a flow chart illustrating a method of generating a template. In the sections herein below, the FPD would be referred to as the detector.

The size of the template is chosen such that the template would be invariant for temperature inhomogeneities over the detector. This is because temperature variations over the template as a function of detector orientation would be low if the size of the template is appropriately chosen.

The first step 100 is to generate a pixellisation image. This step uses at least two gain maps that may be divided pixelwise to obtain a floating-point image, also known as the pixellisation image. Apart from the residue of x-ray quantum noise, the pixellisation image should ideally be a flat image, meaning that there is no variation in the grey level values across the different pixels.

For generating the pixellisation image, at least two homogeneous exposures or image acquisitions are performed on the detector at two different detector operating temperatures. The homogeneous exposure is the time average of a sequence of homogeneous exposures to reduce the x-ray quantum noise. This generates an image, designated by the term “gain map”, for each of the gain calibrations. In one embodiment, the calibrations may be performed at the lowest and highest detector operating temperatures. By this, the characteristic gain drift patterns would have the maximum amplitude. However, any suitable temperature range between the permissible minimum and maximum detector operating temperatures may be appropriately chosen. The gain calibrations can be performed based on the average of multiple images at high doses to reduce the amount of plain x-ray quantum noise in the pixellisation patterns. Optionally, a separate gain calibration for performing a regular correction of detector sensitivity variations can be done.

Optionally, when a defect map of the detector is available, defect exclusion 150 may be done on the pixellisation image to avoid image statistics to be disturbed by defective pixels on the detector. For example, sensitivity variations beyond 20% may be excluded. In another embodiment, additional pre-filtering can be done to improve detection of defective pixels. Further, defect correction 160 can also be performed on the detector to reduce or remove any defects in the pixels. Commonly known detector defect correction methods may be employed to achieve the desired effect.

The next step 200 is to determine local variance of grey values at each pixel position on the pixellisation image. The discarded pixels from the defect detection (when performed) should be excluded from this step. In one embodiment, local variance is calculated at each pixel position in a 5×5 sub-window of the pixellisation image. The choice of the sub-window size may be made to allow for reliable detection of pixellisation while having a reasonable amount of spatial resolution.

The next step 300 is to determine pixellised and non-pixellisation regions from the pixellisation image. To do this, a suitable threshold level must be determined. In one embodiment, rank-order filtering may be used. Consider for example, when pixellisation is assumed to not exceed beyond 40 pixels from the edge of the detector having 1400×1400 pixels. In this case, the worst-case amount of pixellisation pixels can be calculated to be less than 12% for the assumed detector size. While pixellisation was assumed to be present only around the edges of the detector, it is possible for the pixellisation to be present anywhere or all over the detector. The percentage representing the amount of pixellisation pixels will change with respect to size of the detector and the total number of pixels present. In the discussion below, for the sake of clarity, the assumption that pixellisation does not extend beyond 40 pixels from the edge of the detector is considered.

By finding the variance level at which 80% of the pixels has a lower variance level, the pixels that are free of pixellisation can be found. These pixels can then be used to determine the statistics of the non-pixellised pixels. In one embodiment, if the pixel at place coordinate (i, j) of the variance image has a value of Pᵥₐᵣ(i, j) then the spatial mean value of standard deviation can be determined from the formula:

Spatial mean value of standard deviation

$$( M σ ) = \frac{\sum \sqrt{P var ( i , j )}}{n}$$

for all pixels with Pᵥₐᵣ(i, j)<Pᵥₐᵣ(80%) and (i, j)∉defect_pixels and where n is the number of non-defect pixels with a variance less than Pᵥₐᵣ(80%).

The standard deviation of the local standard deviation values is calculated as follows:

$$Standard deviation ( σ ) = \sqrt{( \sum \frac{P var ( i , j )}{n} ) - {( M σ )}^{2}} ,$$

for all pixels where the same conditions apply, where Mσ is the spatial mean value of the standard deviation.

A suitable pixellisation threshold that distinguishes between the pixellised and the non-pixellised regions can be found using the formula:

Threshold Tₚᵢₓₑₗₗᵢₛₐₜᵢₒₙ=(Mσ+F.σ)², where F is a multiplier for the standard deviation value. For a normal distribution, a suitable value for the multiplier F is in the range of 3 to 4. In other embodiments, the threshold value can be appropriately found using techniques known to a person skilled in the art.

Once the threshold value is found, a binary image can be generated 400. This means that each of the pixels has either a NULL value or non-NULL value. In one embodiment, the non-NULL pixels are denoted as being the pixellised pixels. In other embodiments, the NULL pixels can also be denoted as the pixellised pixels. However, appropriate modifications to the formulae need to be made. It should be noted that in the binary image, all pixels are marked based on the threshold value. Pixels that exhibit pixellisation are marked differently from the pixels that do not exhibit pixellisation. The manner of marking the pixels based on the presence or absence of pixellisation is not limited in any particular way. Any suitable distinguishing mark may be used.

Optionally, maximum variance thresholding 450 can also be done. When a high quality defect map of the detector is available, this step may not be required. One advantage of this step is that pixels having unrealistically high pixellisation variance can be excluded. This may be considered as an extra defect detection step.

In the next step 500, the obtained binary image is processed to reduce the number of connected pixellisation regions. In one embodiment, the process may include morphological operations to remove narrow horizontal pixellisation regions (during an opening operation in the y direction) and narrow vertical pixellisation regions (during an opening operation in the x-direction). Further, the morphological operations can also include an opening operation along the x-y direction. Other types of morphological operations may also be performed. For example, dilation operation may be performed to remove tiny template holes and/or to add one or a few pixels around template contours. As will be appreciated by a person skilled in the art, there are many different ways of smoothing a binary region. The morphological operations represent one such way. However, any other suitable method can be employed.

In one embodiment, after the morphological operations are complete, the defect map can be merged with the pixellisation image. All defective pixels and all pixels excluded from the pixellisation detection step (described previously) are set to NULL. This enables the pixellisation correction process to exclude such pixels from the correction process. One advantage of excluding such pixels is the elimination of such pixels from dominating the correction process. The pixellisation image can then be combined with the binary image.

In another embodiment, when all the morphological operations are complete, the binary image can be combined with the pixellisation image. One way of combining the two images include setting of all pixels in the pixellisation image to NULL where the corresponding pixels in the binary image are a NULL. By this, all the non-NULL pixels in the resulting pixellisation image have the required data for forming the pixellisation templates.

The resulting pixellisation image is then sub-divided to form pixellisation templates, more commonly referred to herein below as the templates. In one embodiment, the result of the method of generating a template may result in a single template. In other embodiments, multiple templates may be the generated. In yet another embodiment, the set of templates generated may also be combined to form a single template.

FIG. 2 illustrates a flow chart of a method of image correction of an image, such as a clinical image, having one or more image artifacts. In the sections that follow, the method of image correction is described for a clinical image. However, a skilled person may apply the method to correct or remove artifacts from any image as required. The method also uses one or more templates for the one or more image artifacts found on the clinical image. In one embodiment, the one or more templates may be generated by the method as described herein above. In other embodiments, the templates may already be present and the method simply accesses these templates and uses the templates for the image correction.

In the discussion herein, the method of image correction will describe an act of using the template 600. It must be construed that “using” can mean one of either generating the template during the process of the method of image correction or accessing the template that was generated previously and stored in a database.

Once the template is accessed, the template can be subjected to a convolution operation 650 with a large smoothing kernel. It can be considered as a local averaging process. The convolution step serves as a low-pass filtering step and is used for eliminating any inadvertent offset or weak gradient that may be present in the template data. The low-pass result, with only the offset and gradient information, is subtracted from the template data to provide a desired template (T_{AC}) to be used for image correction. However, the template can be directly used as the desired template (T_{AC}) for the image correction without performing any convolution with the large kernel.

Once the desired template T_{AC} is obtained, a high pass filtering is performed 700 on the template as well as the clinical image. As described previously, pixellisation has its major contributions in the high end of the frequency spectrum. The high pass filtering reduces the influence of the image contents in the image correction method. In one embodiment, the high pass filtering may be performed only on the clinical image. In other embodiments, the high pass filtering can be done on both the template as well as on the clinical image. The result of this step is that the signal transfer of the pixellisation will be identical in the processing of both the template as well as the clinical image.

The image correction method further involves a step of normalization 800. An advantage of this step 800 is that when the image contrast is high at the position of the template, the effectiveness of the correction can be increased. It is known that sensitivity drift is a multiplicative phenomenon. For example, in bright areas of the image, the amplitude of the pixellisation will be higher. The amplitude scales linearly with the local image intensity in the bright areas. The template typically contains no modulation because it is generated from homogeneously exposed images. Therefore, in order to bring the clinical image to a uniform level of the template, the modulation of the pixellisation in the clinical image needs to be removed. Dividing the clinical image by a signal that is proportional to local image intensity achieves this. Low-pass filtering of the clinical image data provides the signal (P_{LP}). During the generation of the template, such type of low-pass filtering can also be done on the template to correct for any non-homogeneity in the template.

The template and the clinical image can now be considered as two one-dimensional vectors of equal size. It may be advantageous to divide all the pixels of the template by the vector length of the template vector. In other words, the template vector is normalized to unity length. Once a scalar product is calculated using the template vector and the clinical image vector, the template vector is multiplied with the result of the scalar product calculation in order to match the length of the template vector with that of the image data.

The template vector length can be calculated by the formula

$$\vert {T}_{HP} \vert = \sqrt{{\sum }_{i , j} {( {T}_{HP} ( i , j ) )}^{2}}$$

for all template pixels T (i, j)≠0. The scalar product, including the calculations for normalising the template vector to a unit vector, can be calculated by the formula:

$$α = {\sum }_{i , j} \frac{{T}_{HP} ( i , j ) \cdot {P}_{HP} ( i , j )}{\vert {T}_{HP} \vert}$$

for all template pixels T(i, j)≠0.

Here, P_{HP} (i, j) is the corresponding pixel at place coordinates (i, j) in the high-pass filtered clinical image. When the template and image data are uncorrelated, the result of the scalar product will be almost zero, because both vectors consist of signed numbers with a mean value of zero. The scalar product calculation is one possible way of determining a correlation between the template and the image.

Each pixel of the normalized template vector can now be scaled by factor α to match the pixellisation level of the template with the pixellisation level of the clinical image. During the scalar product calculation 900, the division by the image vector length is not performed. Therefore, there is no need to multiply with this vector length. Factor α is a factor, which is fit to match the normalised vector T_{HPN} with vector P_{HP}. The factor F that is fit to be applied to vector T_{HP} can be obtained by dividing a by an extra term |T_{HP}|. An advantage of this determination is that factor F can now be calculated without taking a square root.

$$F = \frac{{\sum }_{i , j} {T}_{HP} ( i , j ) \cdot {P}_{HP} ( i , j )}{{\sum }_{i , j} {( {T}_{HP} ( i , j ) )}^{2}}$$

for all template pixels T(i,j)≠0.

Since only a linear filtering operation has been performed to go from the original template data T to the high-pass filtered data T_{HP} and similarly to go from the original image data P to the high-pass filtered image data P_{HP}, the same factor F must be applicable to the pixellisation data from the template data T to bring it to the pixellisation level of the clinical image P 1000. Because of the relatively large kernel that was used for the filtering to obtain template T_{AC}, which is free of offset and gradient errors, factor F can also be safely applied to the filtered template data T_{AC}. Therefore F is the factor that will be used to do the correction to provide a template pixellisation image based on the template that contains the modulation present in the clinical image.

The template pixellisation image has no modulation associated with it. As explained previously, the actual level of pixellisation in the clinical image is modulated by the local intensity of the pixellisation. Earlier, during the normalization process, this modulation was removed by a pixelwise division using the result of the low-pass filtering stage. In order to restore the modulation on the template pixellisation image, a pixelwise multiplication of the template pixellisation image is done. The pixelwise factor that gets multiplied is the low-pass information P_{LP} obtained previously. This step 1100 may be considered as being the opposite of the normalization operation performed earlier as it restores the modulation.

The next step 1200 is the image correction of the clinical image. This step removes the pixellisation from the clinical image, since the clinical image includes the image contents with the pixellisation while the template pixellisation image contains only the pixellisation and no image contents. In one embodiment, the image correction can be done by pixelwise subtraction. When the correlation between the pixellisation in the template data and the image data is high, the pixellisation level will decrease to a level below the visibility threshold as in the final image of the schematic diagram, where the correction actually has been performed.

In one embodiment, the aforementioned methods can be implemented on a system, such as an x-ray imaging system. The system can include means to implement the functionalities by having separate modules to implement each of the functionalities of the method. In other embodiments, the various functionalities may be implemented on one or a few modules. The system can also include an operator workstation to enable an operator to provide the system with commands or instructions, to initiate and to end the correction process when the desired correction of the clinical image is achieved. The system can also include a microprocessor and a display device. In another embodiment, the aforementioned methods may be implemented on a stand-alone system. Such a stand-alone system may be connected to an imaging system or to a database containing acquired images.

In another embodiment, a system for performing image correction would include means for performing the image correction as described above. In one embodiment, the system may access the one or more templates from a database. In other embodiments, the system may include means to generate the one or more templates for use in the image correction. In another possible embodiment, the system for generating the one or more templates can form a part of the system for image correction or vice versa.

As will be appreciated by a person skilled in the art, the embodied methods for generating one or more templates for use in image correction and for image correction may be implemented by means of programmed instructions, such as in the form of computer code. Such code may be comprised in a tangible, computer readable media. In one possible embodiment, the code may be stored directly on a system implementing the methods described above. In another embodiment, the code may be contained in the tangible media and fed into the system. The media may include optical or magnetic media, where the code may be stored appropriately. Examples of such computer media include CDROMs, DVDs, flash memory cards, computer hard drives, floppy disks etc.

1. A method of generating a template of at least one artifact for use in image correction, comprising:
generating (100) a pixellisation image from at least two gain maps, each gain map generated at a different detector temperature;
calculating (200) a local variance of grey values at each pixel position on the pixellisation image;
determining (300) pixelwise, pixellised and non-pixellisation regions on the pixellisation image;
generating (400) a binary image of the pixellisation image based on the determined pixellisation and non-pixellisation regions; and
generating (500) a template based on the pixellisation image and the binary image.

2. The method of claim 1, including performing (150) an exclusion of defect pixels on the pixellisation image based on a defect map prior to calculating the local variance.

3. The method of claim 1, including performing a defect correction (160) on the pixellisation image.

4. The method of claim 1, wherein generating the template includes setting pixel values on the pixellisation image to null for each pixel determined to be defective or designated as the non-pixellisation region.

5. The method of claim 1, including sub-dividing the formed template into separate pixellisation templates for use in the image correction.

6. A method of image correction, comprising:
using (600) a template of at least one artifact in an image requiring correction;
generating (900) a scalar product based on the template and the image;
obtaining (1000) a match between the template and the image based on the scalar product; and
correcting (1200) the image based on the obtained match.

7. The method of claim 6, wherein the step of using the template comprises generating the template or accessing the template from a database.

8. The method of claim 6, comprising normalizing (800) at least one of the template and the image to remove effects of modulation in the template or the image prior to generating the scalar product.

9. The method of claim 8, including restoring (1100) the effects of the modulation after determining the match between the template and the image.

10. A tangible, computer-readable medium for use with an imaging procedure, comprising:
code adapted to generate (100) a pixellisation image from the at least two gain maps;
code adapted to calculate (200) a local variance of grey values at each pixel position on the pixellisation image;
code adapted to determine (300) pixelwise, pixellisation and non-pixellisation regions on the pixellisation image;
code adapted to generate (400) a binary image of the pixellisation image based on the determined pixellisation and non-pixellisation regions; and
code adapted to form (500) a template based on the pixellisation image and the binary image.

11. A tangible, computer-readable medium for use with an imaging procedure, comprising:
code adapted to use (600) a template of at least one artifact in an image requiring correction;
code adapted to generate (900) a scalar product based on the image and the template;
code adapted to determine (1000) a match between the template and the image based on the vector product; and
code adapted to correct (1200) the image based on the match.

12. A system for generating a template for use in image correction, comprising:
means to generate (100) a pixellisation image based on at least two gain maps;
means to calculate (200) a local variance of grey values at each pixel position on the pixellisation image;
means to classify (300) content at each pixel position into pixellisation or non-pixellisation regions;
means to generate (400) a binary image based on the classified pixellisation and non-pixellisation regions; and
means to generate (500) a template based on the binary image and the pixellisation image.

13. A system for correcting an image, comprising:
means to use (600) a template of at least one artifact in a image requiring correction;
means to determine (900) a scalar product based on the template and the image;
means to determine (1000) a match between the template and the image based on the scalar product; and
means to correct (1200) the image based on the match.
